SEQUENCE LISTING (16 sequences)

SEQ ID NO: 1 - DNA, 20 bp, Unknown
  Synthetic oligonucleotide
tgctaggtttcctccctttc

SEQ ID NO: 2 - DNA, 28 bp, Unknown
  synthetic oligonucleotide
ncgatgctaggtttcctccctttgtcgn

SEQ ID NO: 3 - DNA, 28 bp, Unknown
  synthetic oligonucleotide
ncgatgctaggtttcctccctttgtcgn

SEQ ID NO: 4 - DNA, 28 bp, Unknown
  synthetic oligonucleotide
gcgactgactgccaactatgaacatcgc

SEQ ID NO: 5 - DNA, 20 bp, Unknown
  synthetic oligonucleotide
ctgactgccaactatgaaca

SEQ ID NO: 6 - DNA, 20 bp, Unknown
  synthetic oligonucleotide
caaagggaggaaacctagca

SEQ ID NO: 7 - DNA, 20 bp, Unknown
  synthetic oligonucleotide
caaagggaggtaacctagca

SEQ ID NO: 8 - DNA, 20 bp, Unknown
  synthetic oligonucleotide
caaagggaagtaacctagca

SEQ ID NO: 9 - DNA, 43 bp, Unknown
  Part of Mam gene sequence (GenBank Accession No. AF015224)
cggatgaaactctgagcaatgtctgcagttctgtgagccaaag

SEQ ID NO: 10 - DNA, 54 bp, Unknown
  Artificial oligonucleotide
ncaagccggatgaaactctgagcaatgtctgcagttctgtgagccaaagcttgn

SEQ ID NO: 11 - DNA, 26 bp, Unknown
  synthetic oligonucleotide
gcgagcggcggaaaagccatcgtcgc

SEQ ID NO: 12 - DNA, 26 bp, Unknown
  synthetic oligonucleotide
gcgagcaacgagagagccgtcgtcgc

SEQ ID NO: 13 - DNA, 18 bp, Unknown
  Synthetic Oligonucleotide
cgatggcttttccgccgc

SEQ ID NO: 14 - DNA, 28 bp, Unknown
  synthetic oligonucleotide
ncgatgctaggtttcctccctttgtcgn

SEQ ID NO: 15 - DNA, 28 bp, Unknown
  synthetic oligonucleotide
ncgactgactgccaactatgaacatcgn

SEQ ID NO: 16 - DNA, 20 bp, Unknown
  synthetic oligonucleotide
tgctaggtttcctccctttg

(19) United States
(12) Patent Application Publication
(10) Pub. No.: US 2006/0105974 A1
(43) Pub. Date: May 18, 2006

(54) Conjugates of photosensitizers and oligonucleotides for selective photochemiotherapy

(76) Inventors: Norbert Lange, Nyon (CH); Hubert Van Den Bergh, Goumoens-La-Ville (CH)

Correspondence Address: HOWSON AND HOWSON, SUITE 210, 501 OFFICE CENTER DRIVE, FT WASHINGTON, PA, 19034, US

(21) Appl. No.: 10/520,094

(22) PCT Filed: Jul. 4, 2003
(86) PCT No.: PCT/EP03/08052

§ 371 Date: Aug. 26, 2005

(30) Foreign Application Priority Data
Jul. 4, 2002 (GB) 0215534.9

(51) Int. Cl. A61K 48/00; C07H 21/04

(52) U.S. Cl. 514/44; 536/23.1; 604/20; 977/738

(57) ABSTRACT

This invention relates to oligonucleotide targeting agents and their use in the treatment of diseased cells by selective photochemotherapy (PCT). PCT is a method of treating human diseases and disorders, bacteriological indications and other pathological conditions. PCT is based on the topical or systemic application of a photosensitizing agent, a precursor or pro-drug thereof, which preferentially accumulates in the target tissue. Irradiation of the photosensitizing agent located in the target tissue with electromagnetic radiation of an appropriate wavelength and the interaction of the thus excited photosensitive moiety with oxygen leads to tissue damage and destruction of the irradiated areas.

This invention relates to oligonucleotide targeting agents and their use in the treatment of diseased cells by selective photochemotherapy.

Photochemotherapy (PCT) is a method of treating human diseases and disorders, bacteriological indications and other pathological conditions. Furthermore, PCT has been used for cosmetic purposes such as hair removal and skin re-surfacing. PCT is based on the topical or systemic application of a photosensitizing agent, a precursor or pro-drug thereof, which preferentially accumulates in the target tissue. In addition of the photosensitizing agent located in the target tissue with electromagnetic radiation of an appropriate wavelength and the interaction of the thus excited photosensitive moiety with oxygen leads to tissue damage and destruction of the irradiated areas. The therapeutic effect is dependant on the presence of each of the three components involved in the PCT process, electromagnetic radiation, a photosensitive moiety, and oxygen.

The first photosensitizing agent used for the treatment of cancer was hematoporphyrin derivative (HpD). HpD is a complex mixture of porphyrin dimers and oligemers involving ether, ester, and other linkages. Although HpD and its commercial variants have been used extensively in experimental clinical work these first generation photosensitive moieties have at least three important disadvantages. Firstly, they lack selectivity for the target tissue and cause prolonged skin photosensitization due to slow body clearance. Secondly, the absorption in the red wavelength region, where light penetration into the tissue is favored, is relatively weak. Thirdly, they are not well-defined mixtures that are difficult to reproduce.

Various photoactive compounds have been described, including improvements on HpD such as those disclosed in U.S. Pat. Nos. 5,028,621, 4,866,168, 4,649,151 and 5,438,071. The use of pheophorbides was disclosed in U.S. Pat. Nos. 5,198,460, 5,002,962, and 5,093,349. The use of bacteriochlorins was disclosed in U.S. Pat. Nos. 5,173,504, and 5,171,747. In addition, the use of phthalocyanine dyes in PCT was described in U.S. Pat. No. 5,166,197. The use of green porphyrins in PCT was disclosed in U.S. Pat. Nos. 4,883,790, 4,920,143 and 5,171,749. Furthermore, conjugates of chlorophyll and bacteriochlorophylls were disclosed in U.S. Pat. No. 6,147,195.

Due to the drawbacks of conventional photochemotherapeutic agents, research has focused on the development of more potent photosensitizing agents. Despite considerable research efforts in this field, the ideal photosensitive moiety has not been found yet. However, in view of the huge diversity of human disorders and abnormalities this is not surprising. Thus, research in the field of PCT has switched to the development of more selective, targeting photosensitive moieties, in part based on the recent progress made in molecular biology and biochemistry. Following concepts of controlled drug delivery, one approach is based on the covalent coupling of a photosensitizing moiety to a carrier unit that specifically binds to proteins found in abundance in cells associated with the corresponding disease. Typical examples for such targets include antigens, cell surface receptors and cell adhesion molecules. Since the characteristics of tumor selectivity are not further dominated by the pharmacokinetic properties of the photosensitizing agent itself, the properties of the photosensitizing agent can be adapted with respect to tissue optics, singlet oxygen quantum yield and the clinical situation.

Recently, single chain antibody fragments (scFv) coupled to the photosensitive moiety Tin(IV) chlorin e6 have been used to induce selective photothrombosis in experimental animal models used for angiogenic research. However, most PCT targeting agents address specific cell functions associated with angiogenesis, as for example, in advanced cancers Targeting photosensitive moieties which have been coupled to antibodies as specific carrier moiety have unfavorable pharmacokinetic properties including provoking reactions of the immune system, or lacking penetration into the tumor mass. Furthermore, due to the short lifetime of reactive oxygen species (ROS) in biological tissue and consequently their limited radius of action, targeting of functions expressed on the cell surface might significantly reduce the phototoxic efficacy of the targeting photosensitizer. From this point of view, using “cargo” type receptors that serve to deliver metabolic substrates to the target appears more promising. However, this class of receptors which includes the insulin receptor, the low density lipoprotein receptor and the transferrin receptor are often not sufficiently specific and cannot be used for a wide range of diseases.

The human genome project (HGP) has made considerable progress in the identification of specifically expressed gene patterns in a variety of human diseases and disorders and is constantly discovering additional ones. On the basis of the outcome of the HGP, gene therapy seeks to cure these diseases through a number of different strategies. Some of these strategies are aimed at the elimination of disease-associated cells by either improving targeted delivery of toxic drugs or by partial correction of the genetic code that characterizes the given disease. Another strategy is to genetically alter cells to promote disease elimination through the modulation of the immune system. Furthermore, in an additional approach gene expression can be modulated by the exogenous administration of oligonucleotides complementary to a segment of target mRNA which upon hybridisation to the target mRNA sequence blocks translation and thus protein formation.

However, the therapeutic efficacy of these so-called antisense oligonucleotides is limited. In this regard unmodified phosphodiester oligonucleotides become rapidly metabolized both intracellularly and in the blood plasma. Several strategies to avoid this problem have been developed including chemical modification of the bases, the sugar moieties and the phosphodiester backbone. Constructs, in which non-bridging oxygen atoms are replaced by sulfur atoms to give a phosphorthioate oligonucleotide have shown to dramatically increase the resistance against nuclease-mediated decomposition. Furthermore, due to the poor penetration of these compounds through biological barriers such as cellular membranes relatively high concentrations of antisense therapeutics have to be used resulting in non-specific toxic effects. In addition, the presence of a g-quartet, this is a sequence of four successive guanine bases in the antisense sequence, generate a stacked secondary structure that can prevent hybridization, or bind to fibroblasts and heparin-related growth factors. Most of these problems can be overcome by specific chemical modification of one or more components of the antisense compound. However, such modifications can reduce the affinity of the oligonucleotide for the complementary target sequence leading to inefficient suppression of the mRNA-mediated translation into polypeptide chains. Finally, antisense drugs have to be applied aver long periods to induce the wanted therapeutic effects, thus increasing the risk of side effects.

The present invention specifically targets over-expressed nucleic acid sequences thereby offering a general therapeutic methodology for a wide range of human diseases, disorders, and abnormalities. The invention is based on the observation that the phototoxic efficacy of several photosensitizing constructs is strongly reduced if the construct is in close proximity to a molecule that effectively quenches the triplet state. Another possibility is the presence of a molecular group that hinders the collisional energy transfer between the photosensitive moiety and a third molecule such as molecular oxygen.

Similar constructs related to this invention are well-known in the art. However, their use has been limited to diagnostic uses. These compounds which were developed for DNA/RNA analysis using fluorescence-based methods are often referred as “molecular beacons”. Molecular beacons are fluorescently labeled uni- or bimolecular oligonucleotides that upon excitation fluoresce in the presence of the target sequence. Typically these compounds consist of an oligonucleotide with a fluorescent dye tagged to one terminus and a quenching dye to the other. These compounds form a stem-and-loop structure when not hybridized to a target. The loop portion of the molecular beacon can report the presence of a specific complementary nucleic acid sequence. The stem consists of complementary base pairs that keep the two dyes which are linked to the termini of the molecular beacon close together causing quenching of the fluorophore's fluorescence by fluorescence resonance energy transfer (FRET). In the presence of a target nucleic acid sequence, the molecular beacon undergoes a conformational change that forces the stem to separate, resulting in restoration of the fluorescence. Thus, the fluorescence directly indicates the presence of a target.

Depending on the particular situation, quenchers as well as fluorophores can be adapted to the particular problem. Furthermore, nano-scaled gold particles attached to the 5′ terminus of a molecular beacon have been employed as the quencher moiety for a number of different fluorescent dyes. Molecular beacons may differ in loop sequence and loop size as well as in the composition of the stem. Recently, molecular beacons have been designed to study the interaction between oligonucleotides and proteins including single-stranded DNA binding protein and lactate dehydrogenase.

The use of molecular beacons has many advantages over other DNA probes including very high selectivity with single base pair mismatch identification, the capability of studying biological processes in real-time and in vivo and avoiding the inconvenience caused by DNA intercalating reagents.

Psoralen-oligonucleotide conjugates directed against the mRNA sequence of human papillary viruses (HPV) which are associated with cervical cancer have been already described in the literature. Furthermore, U.S. Pat. Nos. 5,587,371 and 5,565,552 disclose conjugates of expanded porphyrins, such as texaphyrins and nucleic acid sequences or analogs thereof. However, the use of such antisense oligonucleotide photosensitive moiety conjugates is limited as they can exhibit photosensitivity without the presence of a target sequence. Additionally, photosensitive oligonucleotides containing a scrambled nucleic acid sequence or one to three base pair mismatches also show significant phototoxicity indicating that such antisense oligonucleotide photosensitive moiety constructs are not specific.

It is an object of the present invention to overcome the drawbacks and limitations of the conventional and/or conjugated photosensitizing agents discussed-above.

### STATEMENT OF INVENTION

According to the present invention there is provided a compound having a structure selected from X—Rₙ-A-Q-Y, Rₙ—X-A-Y-Qₘ, Rₙ—X-A-Qₘ-Y and X—Rₙ-A-Y-Qₘ wherein,

A is a single-stranded nucleic acid sequence, said single-stranded nucleic acid sequence being complementary to a pre-selected target sequence;

R is a photosensitive moiety such that upon irradiation with electromagnetic radiation having a wavelength corresponding to at least one absorption wavelength of R, R interacts through energy transfer with a molecule capable of producing free radicals, to produce free radicals;

Q is a moiety that quenches excited energy states of R;

X and Y are an affinity pair that interact to bring R and Q into close proximity in the absence of said target sequence thus enabling energy transfer between R and Q;

n and m are, independently, integers in the range 1-5; and said compound optionally contains a linker moiety.

Preferably, R has an absorption wavelength of between 300 and 800 nm and is selected from the group consisting of chlorines, chorophylls, coumarines, cyanines, fullerenes, metallophthalocyanines, metalloporphyrins, methylenporphyrins, naphthalimides, naphthalocyanines, nile blue, perylenequinones, phenols, pheophoribes, pheophyrins, phthalocyanines, porphycenes, porphyrins, psoralens, purpurins, quinines, retinols, rhodamines, thiophenes, verdins, xanthenes and dimers, oligomers and derivatives thereof.

Further preferably, Q is selected from the group consisting of a non-fluorescing dye, a fluorophore, a second photosensitizing moiety, a nano-scaled semiconducter or conductor and gold wherein the second photosensitizing moiety is different to R.

Still further preferably, X and Y are selected from the group consisting of complementary nucleic acid sequences, protein-ligand, antibody-antigen and protein-nucleic acid.

Suitably, the linker moiety is selected from the group consisting of linear or branched substituted or unsubstituted alkyl and linear or branched substituted or unsubstituted heteroalkyl groups.

In a preferred embodiment the molecule capable of producing free radicals is molecular oxygen and the free radicals are selected from the group consisting of singlet oxygen and reactive oxygen species.

The compound according to the invention can be unimolecular or bimolecular.

In a preferred embodiment a complex comprising a compound according to the invention is bound to a carrier which increases the internalisation of said compound.

Preferably, the compound is bound to said carrier by electrostatic interaction. In this case the carrier is a polycation and is selected from the group consisting of a histone or polylysine.

Alternatively, the compound is bound to said carrier by covalent interaction. In this case the carrier is a protein or peptide and is selected from the group consisting of an antibody, an antibody fragment, or a cholesterin:

Preferably, the carrier targets a specific cell surface protein wherein the cell surface protein is selected from the group consisting of a low-density lipoprotein receptor, an endothelial growth factor receptor, a fibroblast growth factor receptor, an integrin, an insulin receptor, an epidermal growth factor receptor and a transferrin receptor.

Further preferably, the complex is encapsulated in a lipid mixture, said lipid mixture comprising at least two members independently selected from the group consisting of phospholipids, sterols and cationic lipids.

Typically, the lipid mixture is in the form of liposomes which vary in size from about 50 to 150 nm in diameter.

In a preferred embodiment the compound or complex is associated with at least one pharmaceutically acceptable carrier or excipient.

In a further preferred embodiment the compound or complex is used as a medicament for killing cells by photochemotherapy.

The cells can be prokaryotic or eukaryotic.

Typically, the compound is administered topically, orally or systemically.

Preferably, the cells are involved in neovasculariztion, age related macular degeneration, diabetic retinopathy, artheritis and cancer.

Alternatively, a compound or a complex according to the invention can be used in the preparation of a composition for use in cosmetic treatments.

The invention further provides a method of killing cells by photochemotherapy comprising the steps:

(i) incubating the target cells with an effective amount of a compound or a complex according to the invention;

(ii) allowing sufficient time for the compound to hybridise to a target nucleic acid sequence within the cells; and

(iii) irradiation of the target cells with electromagnetic radiation of a wavelength that corresponds to at least one absorption wavelength of the photosensitive moiety R such that R interacts through energy transfer with a molecule capable of producing free radicals, to produce free radicals.

Preferably, the molecule capable of producing free radicals is molecular oxygen and the free radicals are selected from the group consisting of singlet oxygen and reactive oxygen species.

Further preferably, irradiation with electromagnetic radiation is performed within between 1 minute and 168 hours after incubation with the compound or complex.

Still further preferably, the total fluence of electromagnetic radiation used for irradiation is between 2 J/cm² and 500 J/cm².

The invention also provides kits for preparing a compound or complex according to the invention comprising

(a) one or more affinity pairs;

(b) one or more photosensitizing and quenching moieties; and

(c) one or more target complement sequences;

and a further kit comprising

(a) a compound according to claims 1 to 11;

(b) at least one pharmaceutically acceptable carrier or excipient; and/or

(c) at least one cell surface penetrating assisting agent.

Embodiments of the invention will now be described, by way of example only, and with particular reference to the accompanying drawings, in which:

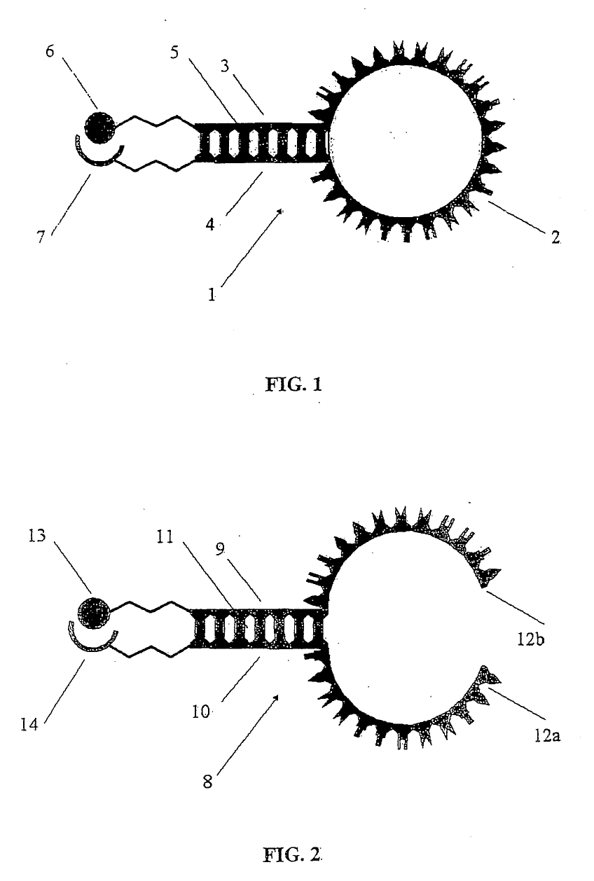

FIG. 1 is a schematic diagram showing the conformation of a unimolecular compound according to the invention;

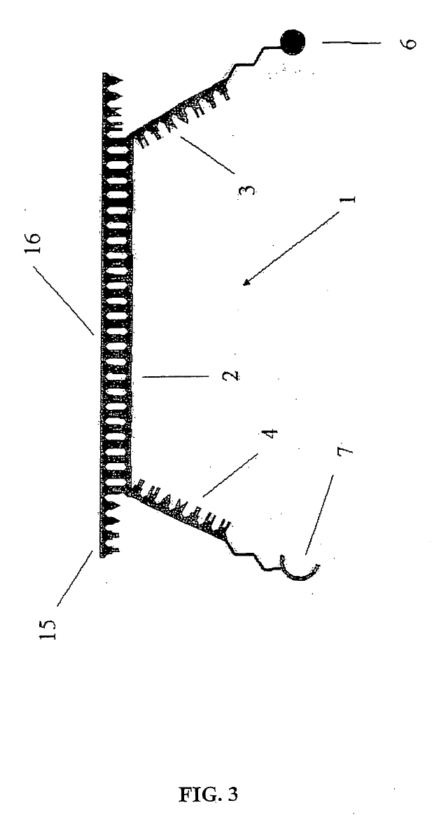

FIG. 2 is a schematic diagram showing the conformation of a bimolecular compound according to the invention;

FIG. 3 is a schematic diagram showing the conformation of the compound when bound to a target nucleic acid sequence;

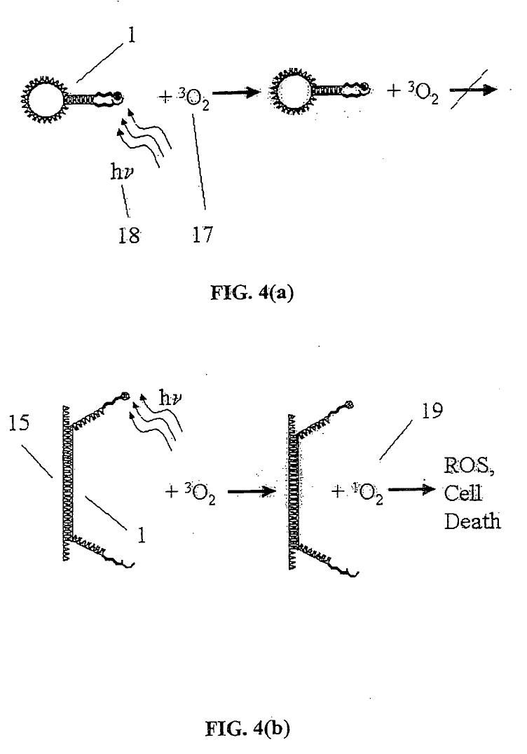

FIG. 4a is a schematic diagram showing the mechanism of action of the compound according to the invention in the absence of the target sequence;

FIG. 4b is a schematic diagram showing the mechanism of action of the compound according to the invention in the presence of the target sequence;

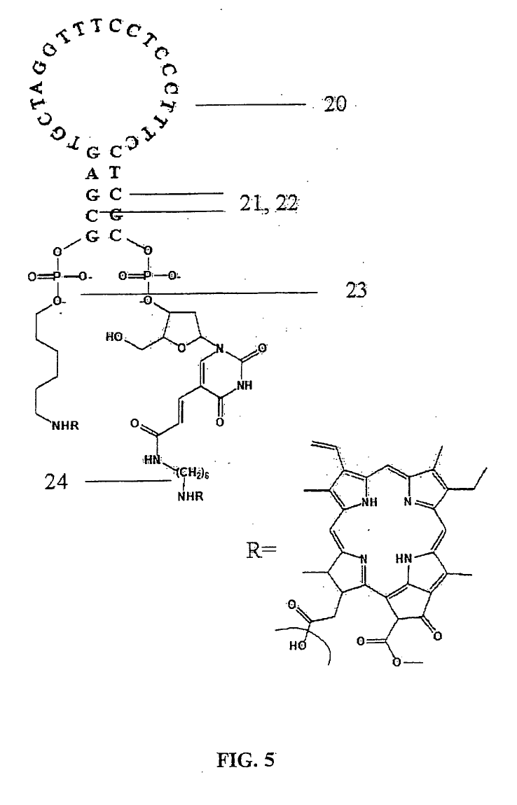

FIG. 5 is a schematic diagram of an exemplary compound according to the invention;

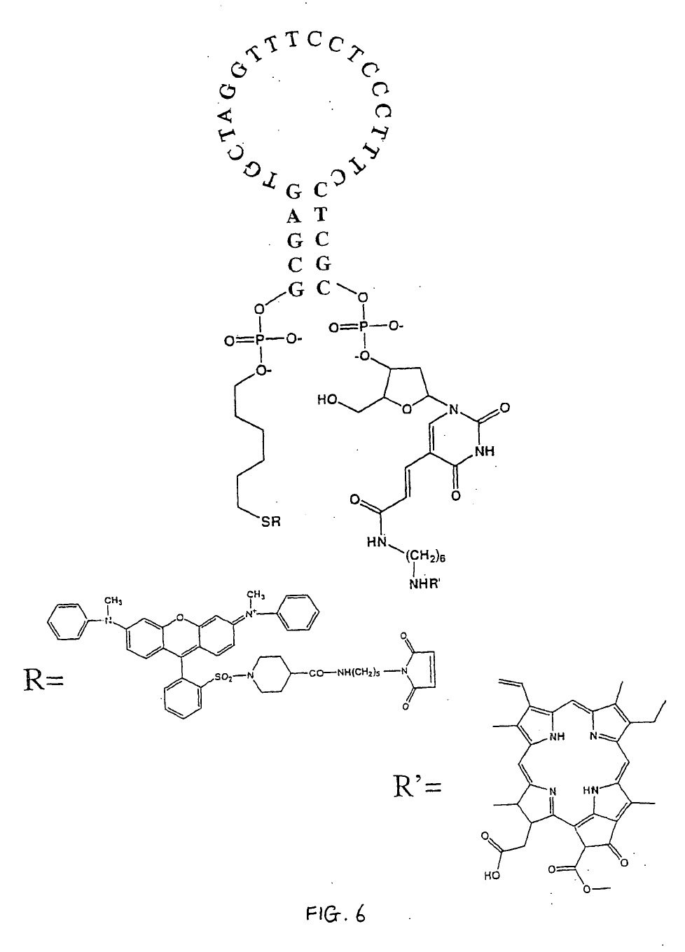

FIG. 6 is a schematic diagram of an exemplary compound according to the present invention;

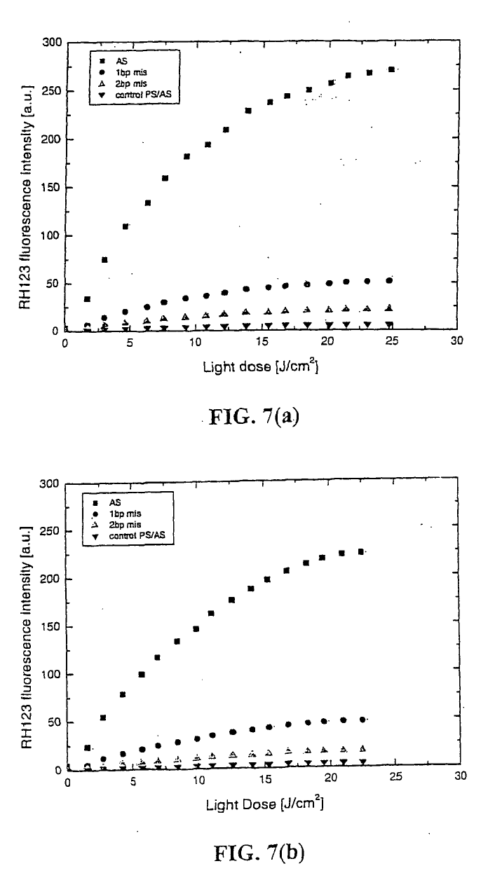

FIG. 7a is a graph showing RH123 fluorescence intensity vs. electromagnetic radiation dose for pheophorbide a labeled compounds according to the invention;

FIG. 7b is a graph showing RH123 fluorescence intensity vs electromagnetic radiation dose for chlorin e labeled compounds according to the invention;

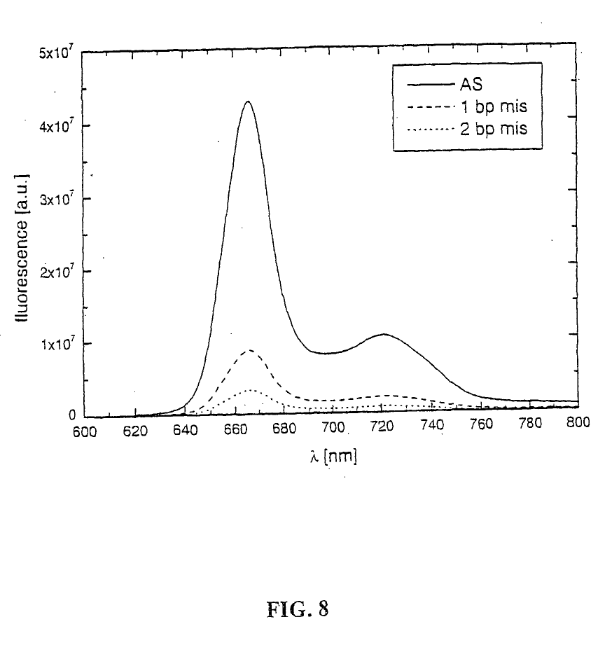

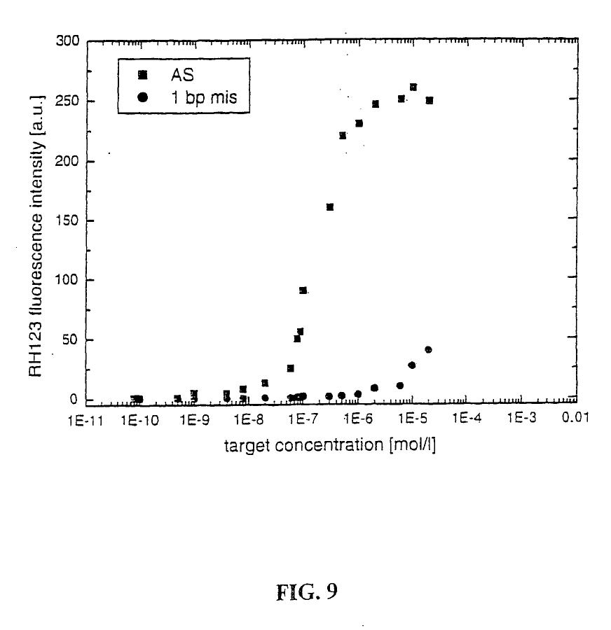

FIG. 8 shows the fluorescence emission spectrum after excitation of the compound according to the invention labeled with pheophorbide a at 405 nm in the presence of a sense oligonucleotide, a sense oligonucleotide with a 1 bp mismatch and a sense oligonucleotide with a 2 bp mismatch;

FIG. 9 is a graph showing the concentration dependence of a compound according to the invention labeled at the 5′ and 3′ termini with a gold particle and pheophorbide a, respectively, vs RH123 fluorescence measured at 529 nm in the presence of a target sequence;

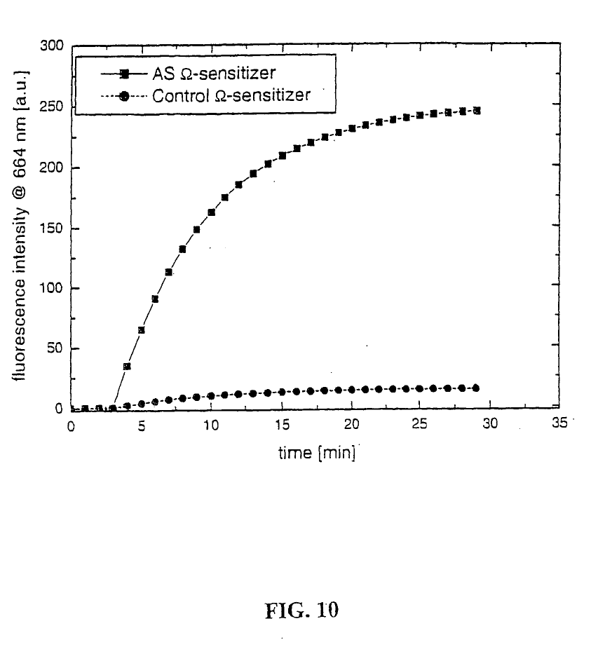

FIG. 10 shows the fluorescence vs time profiles of the hybridisation of an antisense and control compound according to the present invention to an alphaV target sequence;

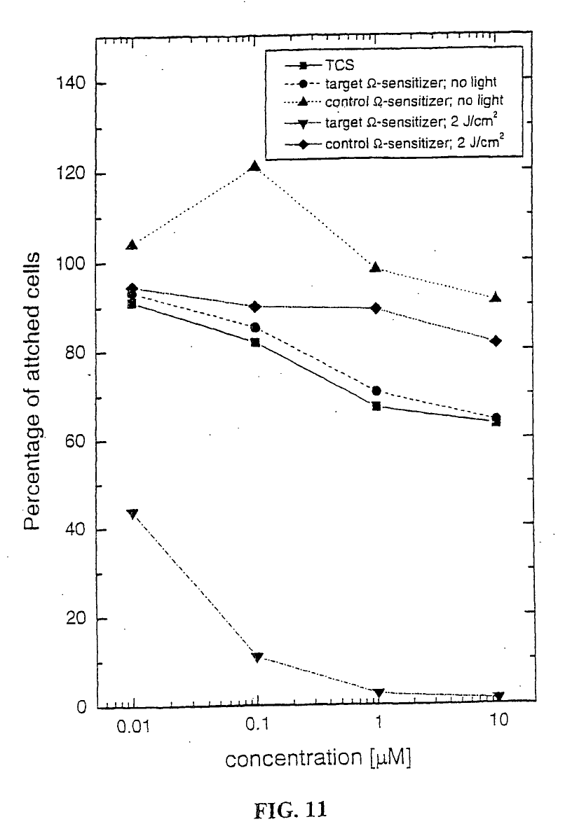

FIG. 11 is a graph showing the percentage of attached cells under different treatment conditions 24 hours after treatment;

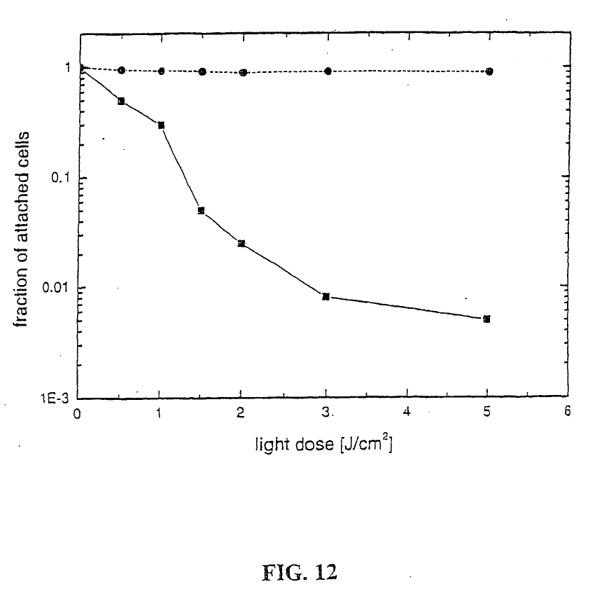

FIG. 12 is graph showing the fraction of attached cells vs. electromagnetic radiation dose;

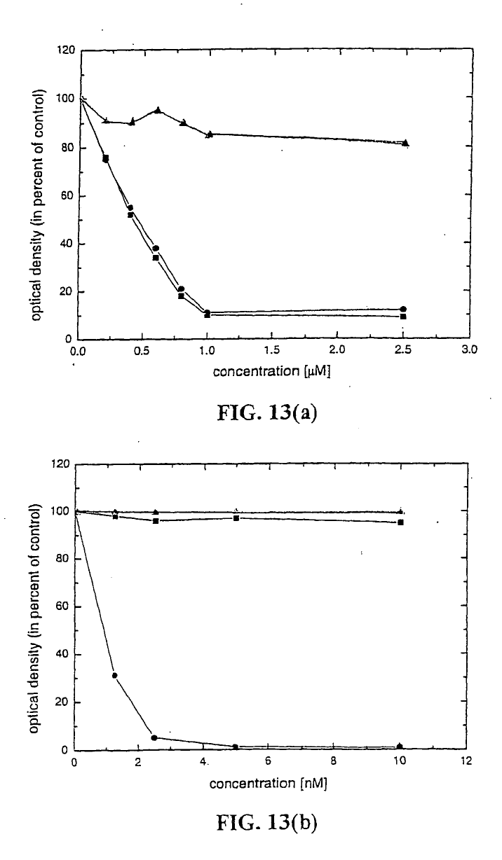

FIG. 13a is a graph showing the inhibition of mitochondrial activity one day after GEO human colon cancer cells were incubated with different compounds according to the invention. The graph is a plot of optical density vs. concentration in μM.

FIG. 13b is a graph showing the inhibition of mitochondrial activity one day after GEO human colon cancer cells were incubated with different compounds according to the invention. The graph is a plot of optical density vs. concentration in nM.

### DETAILED DESCRIPTION OF THE INVENTION

As used herein the term “nucleic acid” means DNA, RNA, singled-stranded, double-stranded, or more highly aggregated hybridization motifs and any chemical modification thereof. Modifications include, but are not limited to, those providing chemical groups that incorporate additional charge, polarizability, hydrogen bonding, electrostatic interaction, and fluxionality to the nucleic acid ligand bases or to the nucleic acid ligand as a whole. The nucleic acid may have modified internucleotide linkages to alter, for example, hybridization strength and resistance to specific and non-specific degradation. Modified linkages are well-known in the art and include, but are not limited to, methylphosphonates, phosphothioates, phosphodithionates, phosphoamidites, and phosphodiester linkages. Alternatively, dephospho-linkages, also well-known in the art, can be introduced as bridges. These include, but are not limited to, siloxane, carbonate, carboxymethylester, acetamide, carbamate, and thioether bridges.

The term “amino acid” as referred herein, means a naturally occurring or synthetic amino acid as understood by persons skilled in the art. It also includes amino acid analogs and mimics.

As used herein, the word “peptides” refers to a polymer of amino acids.

The word “proteins” as used herein, refers to a linear or non-linear polymer of peptides. Proteins include, but are not limited to, enzymes, antibodies, hormones, carriers, etc. without limitation.

As used herein, the word “target”, means any molecule or compound of interest, which is expressed in abundance in the target cell of eucaryotic or procaryotic origin. A target can be, for example, a nucleic acid, a peptide, a protein, a polysaccaride, a carbohydrate, a glycoprotein, a hormone, a receptor, an antibody, a virus, a substrate, a metabolite, a cytokine, an inhibitor, a dye, a growth factor, a nucleic acid sequence, and so on.

Herein the word “alkyl” refers to a branched or linear, saturated or unsaturated, monovalent hydrocarbon radical, generally having between 1-50 carbon atoms, preferably 4-20 carbons and still more preferably between 6-18 carbon atoms. Suitable alkyl radicals are well-known in the art and include, but are not limited to, methylen, methine, and or methyne groups. Branched alkyls have similar motifs to iso-propyl, iso-butyl, tert-butyl, 2-ethylpropyl etc. Alkyls may be modified by replacement substitution or attachment of substituents or substituent of the alkyl. These alkyls are herein referred as substituted alkyl groups. Typical examples for substituents are, for example, alkyl, aryl, acyl, halogen, hydroxy, amino, aminoalkyl, alkoxy, acylamino, thioamino, acyloxy, aryloxy, aryloxyalkyl, mercapto, thia, azo, oxo, saturated and non-saturated cyclohydrocarbons, heterocycles, etc. Furthermore, the term “alkyl” includes structures in which one or more carbon atoms are replaced by oxygen, nitrogen, sulfur, silicium, or phosphor atoms.

The term “functional group” refers to organic substituents including, but not limited to, olefins, acetylenes, alcohols, phenols, ethers, oxides, halides, aldehydes, ketones, carboxyl acids, esters, amides, cyanates, isocyanates, thiocyanates, isothiocyanates, amines, hydrazines, hydrazones, hydrazides, diazo, diazonium, nitro, nitrol, mercaptanes, sulfides, disulfides, sulfoxides, sulfones, sulfonic acids, sulfinic acids, acetals, ketals, anhydrides, sulfates, sulfenic acids, amidines, imides, nitrones, hydroxylamines, oximes, hydroxamic acids, thichydroxamic acids, allenes, ortho esters, sulfites, enamines, amines, ureas, pseudo ureas, semicarbazides, carbodiimides, imenes, azides, azo compounds, azoxy compounds, and nitroso compounds. The synthesis of each functional group is well-known to one of skill in the art.

As used herein, the term “photosensitive moiety” refers to molecules, which upon irradiation with electromagnetic radiation having a wavelength corresponding at least in part to the absorption bands of said molecules interact through energy transfer with another molecule to produce radicals, for example singlet oxygen and other reactive oxygen species. Photosensitive moieties are well-known in the art and include lead compounds, including but not limited to, chlorines, chlorophylls, coumarines, cyanines, fullerenes, metallophthalocyanines, metalloporphyrins, methylenporphyrins, naphthalimides, naphthalocyanines, nile blue, perylenequinones, phenols, pheophoribes, pheophyrins, phthalocyanines, porphycenes, porphyrins, psoralens, purpurins, quinines, retinols, rhodamines, thiophenes, verdins, xanthenes, and dimers and oligomers thereof. The term “photosensitive moiety” as used herein, also includes derivatives thereof wherein at least one of the positions in the lead compound is functionalized by an alkyl, functional group, peptide, protein, or nucleic acid or a combination thereof.

As used herein, the word “quencher” refers to a process by which the excited state energy of an excited molecule or at least a part of it is altered by a modifying group, such as a quencher. If the excited energy modifying group is a quenching group, then one of the excited triplet states or singlet states of the photosensitive moiety is depopulated. If the excited energy modifying group is a large molecule, by which we mean compounds of several hundred Daltons, the energy transfer between the photosensitive moiety and a third molecule or atom is hindered.

Several quenchers are well-known in the art. They include, but are not limited to,

a) non-fluorescing dyes such as DABCYL; DANSYL; QSY-7, BLACK HOLE QUENCHERS, etc.

b) fluorophores, including commercially available fluorescent labels from the SIGMA chemical company (Saint Louis, Mo.), Molecular Probes (Eugene, Oreg.), R & D systems (Minneapolis, Minn.), Pharmacia LKB Biotechnology (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersburg, Md.), Fluka Chemica-Biochemika Analytika (Fluka Chemie AG, Buchs, Switzerland), and Applied Biosystems (Foster City, Calif.), as well as many other commercial sources known to one of skill. Furthermore, those of skill in the art will recognize how to select an appropriate fluorophore for a particular application and, if it not readily available commercially, will be able to synthesize the necessary fluorophore de novo or synthetically modify commercially available fluorescent compounds to arrive at the desired quenching fluorescent label. In addition to small molecule fluorophores, naturally occurring fluorescent proteins and engineered analogues of such proteins are useful in the present invention. Such proteins include, for example, green fluorescent proteins of cnidarians (Ward et al., Photochem. Photobiol. 35: 803-808 (1982); Levine et al., Comp. Biochem. Physiol., 72B: 77-85 (1982)), yellow fluorescent protein from Vibriofischeri strain (Baldwin et al., Biochemistry 29: 5509-15 (1990)), Peridinin-chlorophyll from the Dinoflagellate Syinbiodinium sp. (Morris et al., Plant Molecular Biology 24: 673: 77 (1994)), phycobiliproteins from marine cyanobacteria, such as Synechococcus, e.g., phycoerythrin and phycocyanin (Wilbanks et al., J. Biol. Chem. 268: 1226-35 (1993)), and the like; and

c) Photosensitive moieties (definition see above)

d) Nano-scaled semiconductors, such as quantum dots, nanotubes, and other quantum-well structures; and

e) Nano-sized metal particles such as gold, copper, silver etc.

The term “energy transfer” is well-known to persons skilled in the art, and includes, but is not limited to, transfer of nuclear magnetic energy transfer, transfer of electromagnetic radiation energy, for example fluorescence energy or phosophorescence energy, Förster transfer, or collisonal energy transfer, e.g. energy transfer between an excited photosensitive moiety and a second molecule or transfer of electromagnetic radiation energy into heat, e.g. by internal conversion.

Furthermore, the term “active pair” refers herein to any two or more molecules, preferentially two, that participate in energy transfer. Typically, one of these molecules act as the photosensitizing moiety while the other acts as the excited energy modifying group. In this application there is no limitation with respect to the identity of either groups. All that is required is that the phototoxic activity of the whole changes in the presence of a target. The term active pair is used to refer to a group of molecules that form a complex in which energy transfer occurs. Such complexes comprise, for example two photosensitive moieties, which may be different or equal to each other, a photosensitive moiety and a fluorescent moiety, a photosensitive moiety and a quenching moiety, or any combination of these with multiple groups.

The word “composition” as used herein, means a formulation of compounds or complexes according to the invention with one or more physiologically acceptable carriers or excipients, according to techniques well-known in the art. They may be applied systemically, orally or topically.

Topical compositions include, but are not limited to, gels, creams, ointments, sprays, lotions, salves, sticks, soaps, powders, pessaries, aerosols, and other conventional pharmaceutical forms known in the art. Ointments and creams may, for example, be formulated with an aqueous or oily base with the addition of suitable thickening and/or gelling agents. Lotions may be formulated with an aqueous or oily base and may also contain one or more emulsifying, dispersing, suspending, or thickening agents. Powders may be formed with the aid of any appropriate powder base. Drops may be formed with an aqueous or non-aqueous base containing one or more emulsifying, dispersing, or suspending agent. Alternatively, the compositions may be provided in an adapted form for oral or parenteral administration, including intradermal, subcutaneous, intraperitoneal, or intravenous injection. Alternative pnarmaceutically acceptable formulations include plain or coated tablets, capsules, suspensions and solutions containing compounds according to this invention, optionally together with one or more inert conventional carriers and/or diluents, including, but not limited to, corn starch, lactose, sucrose, microcrystalline cellulose, magnesium stearate, polyvinyl-pyrrolidone, citric acid, tartaric acid, water, water/ethanol, water/glycerole, water/sorbitol, water/polyethylenglycol, propylengycol, water/propyleneglycol/ethanol, water/polyethylenegycol/ethanol, stearylglycol, carboxymethylcellulose, phosphate buffer solution, or fatty substances such as hard fat or suitable mixtures thereof. Alternatively, the compounds according to the invention may be provided in liposomal formulations. Pharmaceutically acceptable liposomal formulations are well-known to persons skilled in the art and include, but are not limited to, phosphatidyl cholines, such as dimyristoyl phosphatidyl choline (DMPC), phosphatidyl choline (PC), dipalmitoyl phosphatidyl choline (DPPC), and distearoyl phosphatidyl choline (DSP), and phosphatidyl glycerols, including dimyristoyl phosphatidyl glycerol (DMPG) and egg phosphatidyl glycerol (EPG). Such liposomes may optionally include other phospholipids, e.g. phosphatidyl ethanolamine, phosphatic acid, phosphatidyl serine, phosphatidyl inositol, abd disaccarides or poly saccarides, including lactose, trehalose, maltose, maltotriose, palatinose, lactulose, or sucrose in a ratio of about 10-20 to 0.5-6, respectively.

The present invention is based, at least in part, on the surprising observation that photosensitizing molecules, coupled to a “hair-pin”-forming olignucleotide having a quenching and/or fluorescent moiety and/or a photosensitizing moiety covalently attached to opposite ends, exhibit phototoxic activity in the presence of a target nucleic acid sequence. In contrast, phototoxicity is totally suppressed or at least limited in the absence of the target nucleic acid sequence.

Compounds according to the invention, herein referred to a “Ω-sensitisers” can consist of either bimolecular pairs of nucleic acid sequences linked together in the absence of target nucleic acid sequence or unimolecular hair-pin forming single strands.

In FIG. 1 the unimolecular form of a Ω-sensitizer is shown. A unimolecular Ω-sensitizer comprises a single-stranded target complement sequence (TCS) 2. The TCS is complementary to a pre-selected target, preferentially complementary to a pre-selected nucleic acid sequence within a nucleic acid target strand. The following description focuses on unimolecular Ω-sensitizers, for convenience. However, persons skilled in the art will understand that similar considerations will apply for the biomolecular version 8 (FIG. 2).

In the unimolecular version 1 the 3′ terminus and 5′ terminus of the TCS are flanked by an affinity pair, herein depicted as oligonucleotide arms 3 and 4. This affinity pair is a pair of moieties that have a certain affinity for each another. Although preferentially composed of complementary nucleic acid sequences, persons skilled in the art can imagine other affinity pairs including, but not limited to, protein-ligand, antibody-antigen, protein-subunits, antigen-antibody fragment, and nucleic acid binding protein binding sites. Sometimes, the use of more than one affinity pair can be used to adapt the binding strength to the desired interaction. The affinity pair reversibly interact to maintain the unimolecular Ω-sensitizer in a closed, phototoxically inactive state in the absence of the target, but the interaction is sufficiently weak that hybridization to the target, preferentially a target nucleic acid sequence, is favored over the interaction of the affinity pair. This interaction is dominated by the laws of thermodynamics and kinetics. It will be evident to persons skilled in the art, that the affinity pair should separate in the presence of a target and not when the unimolecular Ω-sensitizer is non-specifically bound.

Although a number of different affinity pairs can be used, the mechanism by which the Ω-sensitizer undergoes conformational change from a closed to an open state is described herein with an affinity pair of arms consisting of complementary nucleic acid sequences. As shown in FIG. 1, in the absence of a target, the affinity pair hybridizes to form a stem duplex 5 and when the Ω-sensitizer is in this state it is said to be in the closed position. FIG. 3 schematically shows the conformational change of the unimolecular Ω-sensitizer in the presence of a target 15 containing the pre-selected target nucleic acid sequence 16. Due to the favored thermodynamic conditions unimolecular Ω-sensitizer is hybridized to the target sequence 16, forming a relatively rigid double-helix and when the Ω-sensitizer is in this state it is said to be in the open position. For Ω-sensitizers according to this invention, the formation of this double-helix is favored over the hybridization of arms 3 and 4, thus forcing the stem duplex 5 to separate. In the case of bimolecular Ω-sensitizers 8 this helix is in a nicked configuration.

A unitary Ω-sensitizer according to this invention has an active pair, attached to the arms 3 and 4. This active pair as depicted in FIG. 1 as 6 and 7, respectively, and consists of at least one photosensitizing moiety 6 and one quencher moiety 7 allowing effective energy transfer between 6 and 7 in the closed configuration of the Ω-sensitizers 1 and 8, shown in FIG. 1 and FIG. 2 respectively. The photosensitizing moiety 6 can be conjugated to the 3′ terminus or the 5′ terminus of the stem duplex 5. The active pair 6,7 can alternatively be conjugated to other parts of the stem duplex 5 or to the end of the TCS 2.

In FIG. 4 the principle mechanism of selective phototoxic action is depicted. In the absence of a target or the presence of non-target molecules, the unimolecular Ω-sensitizer 1 remains in its closed configuration (FIG. 4a), thus allowing the active pair 6, 7 to interact. Upon irradiation with electromagnetic radiation 18 that is absorbed by the Ω-sensitizer 1, no or only limited energy transfer occurs between the excited photosensitizing moiety 6 and a third molecule 17, herein represented by molecular oxygen. Therefore in the absence of a target sequence the Ω-sensitizer is phototoxically inactive. In contrast, in the presence of a target 15 (see FIG. 4b), the unimolecular Ω-sensitizer 1 undergoes conformational changes, forcing the affinity pair apart. Hence, upon irradiation with electromagnetic radiation 18 that is absorbed by the photosensitizing moiety 6, efficient energy transfer between 6 and a third molecule 17 can occur. In the case that the third molecule is molecular oxygen, highly reactive oxygen in its excited singlet state 19 will be formed. The appearance of sufficient amounts of the product 19 of the energy transfer between the Ω-sensitizer in its open configuration and a third molecule leads to cell death. Therefore in the presence of a target sequence the Ω-sensitizer is phototoxically active. It will be apparent to those skilled in the art that mechanisms other than the energy transfer between molecular oxygen and the phototoxically active Ω-sensitizer may lead to cell death, e.g. the direct formation of other radicals. Furthermore, it is evident that subsequent and/or parallel to the formation of singlet oxygen 19 other reactive oxygen species may be formed that result in the destruction of cells that express the target.

The length of the TCS and the arms are chosen for proper thermodynamic functioning. The length of the TCS can range from about 7 to about 140 nucleotides. The minimum length is limited to the distance where efficient energy transfer between the excited photosensitizing moiety and the quenching moiety is favored over the energy transfer between the excited photosensitizing moiety and a third molecule, preferentially triplet oxygen when the Ω-sensitizer is in its open state. Depending on the treatment conditions the arms can be from about 3 to about 25 nucleotides in length, and are preferably between 4 and 15 nucleotides in length, and more preferably still between 5 and 11 nucleotides in length. It will be understood by persons skilled in the art, that the length of the arms should be adapted to the length of the TCS.

In the case of oligonucleotide sequences functioning as the affinity pair, the upper limit of their length is governed by two complementary thermodynamic criteria. Firstly, it is prefered that the melting temperature of the stem duplex is higher than the treatment conditions, preferentially between 25° C. and 45° C. Treatment conditions between 30° C. and 40° C. are preferred. Furthermore, it is preferred that the melting temperature of the stem duplex is at least 5° C. higher than the treatment conditions, and more preferably 10° C. higher.

Secondly, in view of the thermodynamically balanced conformational change the energy that is released during the formation of the stem duplex should be inferior to the energy that is released during the hybridization of the TCS to the target. Consequently, the arms should be shorter than the TCS.

Although, in FIGS. 1 and 2 the active pairs (6, 7 and 13, 14, respectively) are depicted as photosensitive moiety/quencher pair, other combinations are possible, e.g. photosensitive moiety/photosensitive moiety or photosensitive moiety/fluorescent dye, or photosensitive moiety/quantum dot. However, the pair must be designed so that energy transfer is maximal, when the Ω-sensitizer is in its closed conformation. Furthermore, both photosensitive moiety and quencher must have a functional group that allows conjugation to the TCS/affinity pair construct. The use of multiple quenching moieties with a single photosensitive moiety will increase the efficacy of the energy transfer. A fluorophor as quenching moiety adapted to the spectral characteristics of the photosensitizing moiety will help to localize the Ω-sensitizer during or prior to the treatment. In this case fluorophores with excitation/emission wavelengths that are distinct from those of the photosensitizing moiety are preferred. Optionally, the intrinsic fluorescence of the photosensitizing moiety can be used for the localization or the determination of the distribution of the Ω-sensitizer within the subject treated with the Ω-sensitizer. Alternatively, the Ω-sensitizer can be labeled at any position of the Ω-sensitizer with a luminescent label moiety. Such moieties can be selected from any one of the following categories which are well-known to those skilled in the art: fluorescent label, chemiluminescent label, bioluminescent label, radioluminescent label, electroluminescent label or any combination thereof. Furthermore, the arms of the Ω-sensitizer can be additionally labeled by a luminescent label having a corresponding quencher moiety on the opposite terminus of the Ω-sensitizer. Such constructs are useful to indicate when the Ω-sensitizer is phototoxically active, as shown by the signal emitted by the label after excitation with electromagnetic radiation of an appropriate wavelength.

In a further embodiment, the active pair 6, 7 can be located along the stem duplex 5, rather than at the 5′ and 3′ termini of the affinity pair (FIG. 1). This may increase the interaction of the active pair when the Ω-sensitizer is in the closed conformation. When placing the active pair along the stem duplex, considerations should be given to the helical structure of the stem duplex. In this regard the moieties may be conjugated to the nucleotides staggered along each arm such that when the arms anneal, the moieties of the active pair will be on the same side of the stem duplex helix maximizing the energy transfer between the moieties of the active pair upon irradiation. Sometimes the moieties of the active pair are attached by covalent linkage through spacers, preferentially alkyl spacers (see FIGS. 5 and 6). The nature of this spacer, however, is not critical. The introduction of such a spacer can facilitate the conjugation of each of the moieties of the active pair. In general, the components of the Ω-sensitizer of the invention are linked together through the use of reactive groups, which are typically transformed by the linking process into a new organic functional group. The reactive functional group or groups can be located at any position on an aryl nucleus or on a chain, such as an alkyl chain, attached to an aryl nucleus. When the reactive group is attached to an alkyl, or substituted alkyl chain, the reactive group is preferably located at a terminal position of an alkyl chain.

Reactive groups and classes of reactions useful in practicing the present invention are generally those that are well known in the art of bioconjugate chemistry. Currently favored classes of reactions available are those which proceed under relatively mild conditions. These include, but are not limited to nucleophilic substitutions (e.g., reactions of amines and alcohols with acyl halides, active esters), electrophilic substitutions (e.g., enamine reactions) and additions to carbon-carbon and carbonheteroatom multiple bonds (e.g., Michael reaction, Diels-Alder addition).

Useful reactive functional groups include, for example:

(a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;

(b) hydroxyl groups, which can be converted to esters, ethers, aldehydes, etc.;

(c) haloalkyl groups, wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;

(d) dienophile groups, which are capable of participating in Diels-Alder reactions such as, for example, maleimido groups;

(e) aldehyde or ketone groups, such that subsequent derivatization is possible via formation of carbonyl derivatives such as, for example, imines, hydrazones, semicarbazones or oximes, or via such mechanisms as Grignard addition or alkyllithium addition; sulfonyl halide groups for subsequent reaction with amines, for example, to form sulfonamides;

(g) thiol groups, which can be, for example, converted to disulfides or reacted with acyl halides;

(h) amine or sulfhydryl groups, which can be, for example, acylated, alkylated or oxidized;

(i) alkenes, which can undergo, for example, cycloadditions, acylation, Michael addition, etc;

(j) epoxides, which can react with, for example, amines and hydroxyl compounds; and

(k) phosphoramidites and other standard functional groups useful in nucleic acid synthesis.

More specifically, there are many linking moieties and methodologies for attaching groups to the 5′-or 3′-termini of oligonucleotides. For example, chlorin e6, mono-aspartyl chlorin e6, Tin(IV) chlorin e6, protoporphyrin IX, or pheophorbide a may be linked to the 5′-or 3′-termini of oligonucleotides via a six-carbon-long alkyl linker well-known and commonly used in the art. The introduction of linkers makes the moieties flexible. The flexibility facilitates maximum energy transfer between the photosensitive moiety and the quencher moiety, thus minimizing phototoxicity of the Ω-sensitizer when in the closed conformation. Suitable linkers for the 3′ and 5′ termini are well-known to persons skilled in the art and their length may vary between 1 to 20 carbon atoms. For multiple conjugation to one or both arms of the affinity pair, bifurcated linkers are preferred.

The nucleic acids of the Ω-sensitizer can be synthesized by a number of different approaches including commonly known methods of solid-phase chemistry. Conventionally, the Ω-sensitizers according to this invention are synthesized on an automated DNA synthesizer (e.g. P.E. Biosystems Inc. (Foster Clif, Calif.) model 392 or 394) using standard chemistries, such as phosphoramidite chemistry. When using automated DNA synthesizers, the active pair is preferentially introduced during automated synthesis. Alternatively, one or more of these moieties can be introduced either before or after automated synthesis. Furthermore, labeled nucleotides can be used during automated synthesis. Additional strategies for conjugation to growing or complete sequences will be apparent to those skilled in the art.

Following automated synthesis the reaction products are preferentially cleaved from their support, protection groups are removed and the Ω-sensitizer purified by methods known in the art, e.g. chromatography, extraction, gel filtration, or high pressure liquid chromatography (HPLC). In other embodiments, modified nucleic acids can be introduced, including modified phosphate backbones, and/or modified bases, and/or modified sugar moieties. For example, the oligonucleotide can comprise at least one modified base moiety which is selected from the group including, but not limited to, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylamino methyl-2-thiouridine, 5-carboxymethylaminomethyluracil, dihydrouracil, beta-D-galactosylqueosine, inosine, N-6-isopentenyladenine, 1-methylguanine, 1-methylinosine, 2,2-dimethylguanine, 2methyladenine, 2-methylguanine, 3-methylcytosine, 5-methylcytosine, N-6-adenine, 7-methylguanine, 5-methylaminomethyluracil, 5-methoxyaminomethyl-2-thiouracil, beta D-mannosylqueosine, 5-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methyl ester, uracil-5-oxyacetic acid (v), 5-methyl-2-thiouracil, 3 (3-amino-3-N-2-carboxypropyl) uracil, (acp3) w, and 2,6-diaminopurine.

In another embodiment, the oligonucleotide can comprise at least one modified sugar moiety selected from the group including, but not limited to, arabinose, 2-fluoroarabinose, xyulose, and hexose.

In yet another embodiment, the oligonucleotide can comprise at least one modified phosphate backbone selected from the group including, but not limited to, a phosphorothioate, a phosphorodithioate, a phosphoramidothioate, a phosphoramidate, a phosphordiamidate, a methylphosphonate; an alkyl phosphotriester, and a formacetal or analog thereof. Such modifications will enhance the intracellular, extracellular, and plasma stability of the Ω-sensitizer and are well-known by persons skilled in the art.

A bimolecular Ω-sensitizer 8 according to the present invention can be synthesized following similar strategies to those described above and will be apparent to persons skilled in the art. In the case of bimolecular Ω-sensitizers, two oligonucleotides are synthesized each comprising one half, or approximately one half, of the TCS and one arm separately. Each of these arms is then conjugated to one or more moieties of the active pair either prior to or during synthesis, and are preferentially located at the 5′ and 3′ termini, respectively. On the basis of a unitary bimolecular Ω-sensitizer, unimolecular Ω-sensitizers can be obtained by annealing both parts of the bimolecular Ω-sensitizer, if desired.

Kits containing a stem duplex conjugated with an active pair and instructions to use this kit for the preparation of compounds according to the invention for the treatment of areas containing one or more pre-selected targets are also envisaged. A labeled duplex stem may be supplied and used as unit, with the arms hybridized to each other, such that a linking end of the stem duplex structure comprising the reactive groups is formed. Alternatively, other biochemical linking agents or chemical agents may be present at the 5′ and 3′ linking termini of the labeled stem duplex. Chemical ligation may involve functional groups. Providing one or more TCS with the kit, which can be conjugated to the stem duplex will enable preparation of Ω-sensitizers in situ. This arrangement may be advantageous because one kit can contain several TCS, complementary to targets overexpressed in different diseases, thus the Ω-sensitizer can be adapted to the particular treatment conditions and be more flexible with respect to the pathological condition to be treated. It will be obvious to those skilled in the art that this is particularly the case when mutually exclusive reactions are used for the attachment of each arm portion. In this case, the stem portions may be kept separately for sequential conjugation reactions. Preferably, kits for Ω-sensitizer preparation contain stem portions of between 5 and 20 oligonucleotides in length.

Kits according to the invention may also contain one or more stem portions and instructions for preparing suitable TCS, or appropriate restriction fragments, which can be linked to the stem. A kit may contain multiple stems and TCS varying, e.g. in melting temperature and/or length of the final Ω-sensitizer to be formed. Optionally, kits according to this invention may include enzymes, reagents and other devices so that the user of the kit may easily use the kit for the preparation of Ω-sensitizers directed against a preselected target.

Sometimes it may be difficult to introduce nucleic acids into the cell or to body areas expressing the target and another aspect of this invention is Ω-sensitizers which are bound to effective delivery systems to facilitate intracellular bio-availability of said Ω-sensitizers at levels required for effective PCT both in vivo and in vitro. Such molecular complexes comprise a Ω-Synthesizer according to the invention, complexed or covalently bound to a carrier which is a conjugate of a cell-specific or non-specific binding agent. The complex is administered in a pharmaceutically acceptable solution in an amount sufficient to perform photochemotherapy in the region of interest. The ligand binding molecule includes any cell surface recognizing molecule. It can include any molecule with a specific affinity for a cell surface component. The cell surface component can be those generally found on any cell type. Preferably, the cell surface component is specific to the cell type targeted. More preferably, the cell surface component also provides a pathway for entry into the cell, for the said binding molecule complex or said Ω-Sensitizer attached thereto. Preferably, the conjugation of the Ω-Sensitizer to the ligand molecule does not substantially interfere with the ability of the ligand binding molecule to bind to the cell surface molecule or for entry of the conjugate or the Ω-Sensitizer into the cell. More preferably, the ligand binding molecule is a growth factor, an antibody or antibody fragment to a growth factor, or an antibody or antibody fragment to a cell surface receptor. Alternatively, the ligand can comprise an antibody, antibody fragment (e.g., an F(ab′)2 fragment) or analogues thereof (e.g., single chain antibodies) which bind to the cell surface component, typically a receptor, which mediates internalization of bound ligands by endocytosis. Such antibodies can be produced by standard procedures.

The complex of the Ω-Sensitizer and the binding molecule can be used in vitro or in vivo to selectively deliver Ω-Sensitizer to target cells. The complex is stable and soluble in physiological fluids. It can be administered in vivo where it is taken up by the target cell via the surface-structure-mediated endocytotic pathway.

The binding moiety of the carrier complex performs at least two functions:
(1) it binds Ω-Sensitizer in a manner which is sufficiently stable (either in vivo, ex vivo, or in vitro) to prevent significant uncoupling of the complex extracellularly prior to internalization by a target cell; and
(2) it binds to a component on the surface of a target cell so that the carrier complex is internalized by the cell. Generally, the carrier is made up of a cell-specific ligand and a cationic moiety which are conjugated. The cell-specific ligand binds to a cell surface component, such as a protein, polypeptide, carbohydrate, lipid or combination thereof. It typically binds to a cell surface receptor. The cationic moiety is electrostatically bound to the polynucleotide.

The binding moiety can also be a component of a biological organism including viral particles and cells (e.g., mammalian, bacterial, protozoan).

The bond between the Ω-Sensitizer and the binding ligand can be covalent or non-covalent. A non-covalent bond based on electrostatic attraction between the binding agent and the Ω-Sensitizer provides extracellular stability and is releasable under intracellular conditions. Preferred Ω-Sensitizer-binding agents are polycations that bind the negatively charged nucleic acid strands. These positively charged materials can bind non-covalently with the Ω-sensitizer to form a soluble, targetable molecular complex which is stable extracellularly but which releases the Ω-Sensitizer as a functional (e.g., hybridizable) molecule intracellularly. Suitable polycations are polylysine, polyarginine, polyornithine, basic proteins such as histones, avidin, protamines and the like. A preferred polycation is polylysine (e.g., ranging from 3,800 to 60,000 daltons). Other non-covalent bonds that can be used to releasably link the Ω-sensitizer include hydrogen bonding, hydrophobic bonding, electrostatic bonding alone or in combination such as, anti-poly- or oligonucleotide antibodies bound to poly- or oligonucleotides, and streptavidin or avidin binding to poly- or oligonucleotide containing biotinylated nucleotides.

Alternatively, the complex can be formed by covalent linking of the Ω-Sensitizer and a cell-specific ligand using standard cross-linking reagents which are well known in the art. A preferred linkage is a peptide bond. This can be formed with a water soluble carbodiimide, such as 1-ethyl-3-(3-dimethylaminopropyl) carbodiimide hydrochloride (EDC). Alternative linkages are disulfide bonds which can be formed using cross-linking reagents, such as N-Succinimidyl 3-(2-pyridyldithio)propionate (SPDP), N-hydroxysuccinimidyl ester of chlorambucil, N-Succinimidyl-(4-Iodoacetyl) aminobenzoate) (SIAB), Sulfo-SIAB, and Sulfo-succinimidyl-4-maleimidophenyl-butyrate (Sulfo-SMPB). Strong noncovalent linkages, such as avidin-biotin interactions, can also be used to link cationic moieties to a variety of cell binding agents to form suitable carrier molecules.

The linkage reaction can be optimized for the particular cationic moiety and cell binding agent used to form the carrier. The optimal ratio (w:w) of cationic moiety to cell binding agent can be determined empirically. This ratio will vary with the size of the cationic moiety (e.g., polycation) being used in the carrier, and with the size of the polynucleotide to be complexed. However, this ratio generally ranges from about 0.2-5.0 (cationic moiety ligand). Uncoupled components and aggregates can be separated from the carrier by molecular sieve or ion exchange chromatography (e.g., AQUAPORE®, cation exchange, Rainin).

As mentioned above, a further object of the invention accordingly provides a pharmaceutically acceptable composition comprising a compound or a complex according to the invention, together with at least one pharmaceutical carrier or excipient. It will be apparent to persons skilled in the art that the concentrations of the compounds of the invention depend upon the nature of the compound, the composition, the mode of administration and the patient and may be varied or adjusted as required. For topical application, e.g. concentration ranges from 0.05 to 50% (w/w) are suitable, more preferentially from 0.1 to 20%. Alternatively, for systemic application drug doses of 0.05 mg/kg body weight to 50 mg/kg body weight of photosensitive moiety equivalents, more preferentially 0.1 to 10 mg/kg, are appropriate.

The present invention includes methods, using compounds or complexes according to the invention or any pharmaceutically acceptable composition thereof for therapeutic purposes, preferentially photochemotherapeutic purposes. Diseases or disorders, which may be treated according to the present invention include any malignant, pre-malignant and non-malignant abnormalities responsive to photochemotherapy, including, but not limited to, tumors or other growths, skin disorders such as psoriasis, skin cancer, or actinic keratosis, and other diseases or infections, e.g. bacterial, viral or fungal infections. Methods according to this invention are particularly suited when the disease is located in areas of the body that are easily accessible to electromagnetic radiation, such as internal or external body surfaces. These surfaces include, e.g. the skin and all other epithelial and serosal surfaces, including for example mucosa, the linings of organs, e.g. the respiratory, gastro-intestinal and genito-urinary tracts, glands, and vesicles.

In addition to the skin, such surfaces include for example the lining of the vagina, the endometrium, the peritoneum, the urothelium, and the synovium. Such surfaces may also include cavities formed in the body following excisions or incisions of diseased areas, e.g. brain cavities. Exemplary surfaces using methods according to this invention are listed in Table 1:

TABLE 1
List of some exemplary body surfaces

Skin
Conjunctiva
Linings of the mouth, pharynx, and larynx
Linings of the oesophagus, stomach, intestines, and intestinal
appendages
Linings of the rectum and the anal canal
Linings of the nasal passages, nasal sinuses, nasopharynx
Linings of the trachea, bronchi, and bronchioles
Linings of the ureters, urinary bladder, and urethra
Linings of the vagina, uterine cervix, and uterus
Parietal and visceral pleura
Linings of the peritoneal and pelvic cavities
Dura mater and meninges
Any tumor in solid tissues that can be made accessible to
photoactivating electromagnetic radiation

For persons skilled in the art of PCT it will be apparent that methods according to this invention may also be suitable for the treatment of angiogenesis associated diseases, when the target tissue is vascular endothelial tissue. Typical examples include, but are not limited to an abnormal vascular wall of a tumor, a solid tumor, a head tumor, a neck tumor, a tumor of the gastrointestinal tract, a liver tumor, a breast tumor, a prostate tumor, a lung tumor, a nonsolid tumor, malignant cells of a hematopoietic tissue and lymphoid tissue, vascular lesions, diseased bone marrow, and diseased cells involved in autoimmune and in inflammatory conditions such as rheumatoid arthritis or choroidal neovascularization associated with age-related mucular degeneration. In yet a further method of the present invention, the target tissue is a lesion in a vascular system. It is contemplated that the target tissues a lesion of the type selected from the group consisting of atherosclerotic lesions, arteriovenous malformations, aneurysms and venous lesions. Methods according to this invention may also be used for cosmetic purposes, hair removal, depilation, removing varicoses etc. The present invention may also be useful for the treatment of conditions of protozoan and parasitic origin, particularly acne, malaria and other conditions resulting from parasites. The term “parasite” includes parasites of humans and other animals, including parasitic protozoa (both intracellular and extracellular), parasitic worms (nematodes, trematodes, and cestodes) and parasitic ectoparasites (insects and mites).

The parasitic Protozoa include malarial parasites of humans and/or other animals including but not limited to Plasmodium falciparum, Plasmodium ovale, Plasmodium malaria, Plasmodium vi vax; leishmanial parasites of humans and/or other animals including but not limited to Leishmania tropica, Leishmania major, Leishmania aethiopica, Leishmania brasiliensis, Leishmania guyanensis, Leishmania panamenis, Leishmania peruviana, Leishmania mexicana, Leishmania amazonensis, Leishmania pifanoi, Leishmania garnhami, Leishmania donovani, Leishmania infantum, Leishmania chagasi; trypanosomal parasites of humans and/or other animals including but not limited to Trypanosoma cruzi, Trypanosoma brucei gambiense, Trypanosoma brucei rhodesiense; amoebic parasites of humans and/or other animals including but not limited to Entamoeba histolytica Naeglaria species, Acanthamoeba species, Dientamoeba fragilis; and miscellaneous protozoan parasites of humans and/or other animals including but not limited to Toxoplasma gondii, Pneumocystis carinii, Babesia microti, Isospora belli, Cryptosporidium, Cyclospora species, Giardia lamblia, Balantidium coli, Blastocystis hominis, Microsporidia species, Sarcocystis species. Some of these miscellaneous protozoa cause self-limiting diseases in normal people, but can cause serious problems in HIV patients.

Parasitic nematodes in humans and/or animals include but are not limited to the filarial nematodes Wuchereria bancrofti, Brugia malayi, Brugia timori, Onchocerca volvulus, Loa loa, Tetrapetalonema perstans, Tetrapetalonema streptocerca, Mansonella ozzardi, Dirofilaria immitis, Dirofilaria tenuis, Dirofilaria repens—intestinal nematodes, Ascaris lumbricoides (roundworm), Necator americanus (hookworm), Ancylostoma duodenale (hookworm), Strongyloides stercoralis (threadworm), Enterobius vermicularis (pinworm), Trichuris trichiura (whipworm), Trichostrongylus species, Capillaria philippinensis; tissue nematodes including but not limited to Trichinella spiralis, Anasakis species, Pseudoterranova species and Dracunculus medinensis; parasitic trematodes in humans and/or other animals including but not limited to Schistosoma mansoni, Schistosoma haematobium, Schistosoma japonicum, Clonorchis sinensis, Paragonimus species, Opisthorchis species, Fasciola hepatica, Metagonimus yokogawai, Heterophyes heterophyes and Fasciolopis buski; parasitic cestodes in humans and/or other animals including but not limited to Taenia saginata, Taenia solium, Hymenolepis species, Diphyllobothrium species, Spirometra species and Echinococcus species.

It will be understood that methods using compounds according to this invention may also be useful in sterilization in the food industry and agriculture.

Methods according to this invention employ, in general, several distinct steps. Firstly, a compound, complex or composition according to this invention is applied, preferentially to a mammalian subject. Following administration the area of interest is exposed to electromagnetic radiation in order to achieve a photochemotherapeutic effect. The time period between administration and irradiation will depend on the nature of the compound, the composition, the form of administration and the subject. Time periods between 1 minute and 168 hours are envisaged to be useful in accordance with the present invention. However time periods between 4 minutes and 96 hours are more preferred. Irradiation can be performed using a continuous or pulsed electromagnetic radiation source with electromagnetic radiation doses ranging from between 2 to 500 J/cm². Electromagnetic radiation doses between 5 and 200 J/cm² are preferred. In addition the electromagnetic radiation dose may be applied in one portion or several distinct portions of electromagnetic radiation. It will be understood by persons skilled in the art that the wavelength of electromagnetic radiation used for irradiation must be selected from at least one of the absorption bands of the photosensitizing moiety of said Ω-sensitizer in its phototoxically active configuration. Furthermore, it will be understood by persons skilled in the art, that doubling the corresponding wavelength, using an effect commonly known as “two photon absorption” will also induce the process described above. Conventionally, when porphyrins are used as photosensitizing moieties, they are irradiated with electromagnetic radiation of wavelengths in the region of between 350 and 660 nm. For chlorines this range should be extended to 780 nm, and for phthalocyanines the range should be extended to 800 nm. In particular the highest and lowest absorption bands of the particular photosensitizing moiety are most useful. For example wavelengths in the red region of the spectrum are particularly useful for treating bulky or deeper lying lesions and disease in the retina or the subretina, as well as vascular lesions. Wavelengths in the blue region of the visible spectrum are useful for treating superficial lesions thus preventing side effects including pain, stenosis, occlusion, or necrosis in muscle tissue. However, superficial lesions can aslo be treated with red or green light. Table 2 lists some exemplary photosensitive moieties and their corresponding wavelength regions. The table shows only some examples of useful photosensitizing moieties and should not be understood as limitating.

Methods of irradiation of different areas of the body and methods to bring the electromagnetic radiation to the internal body cavities from light sources including lamps, laser, and light emitting diodes are well known in the art. It will be obvious to persons skilled in the art that alternatively transdermal irradiation can be performed.

The following examples are for illustrative purposes only and are not intended to restrict the invention in any way.

### EXAMPLE 1

Example 1 describes the synthesis of two exemplary Ω-sensitizers with two pheophorbide a or chlorin e6 moieties as active pair, respectively. As depicted in FIG. 5 for pheophorbide a the Ω-sensitizer comprises a 20 mer TCS 20, 5′-TGC TAG GTT TCC TCC CTT TC-3′, directed against the epidermal growth factor receptor (EGFR) mRNA. The TCS is directly flanked by two complementary arms 21 and 22 which form the stem duplex in the absence of the target. Two identical photosensitive moieties R are coupled via two different amino carbon spacers 23 and 24 to the 3′ and the 5′ terminus of the arms 21 and 22, respectively. Oligonucleotides were synthesized using an Applied Biosystems 394 DNA synthesizer (Perkin Elmer, Applied Biosystems Inc., Foster City, USA) and standard phosophoramidite chemistry. They were grown on a controlled pore glass support functionalized with an amine group attached via a ten atom linker (5′-DMT-T(C6 Amino)-Suc-CPG Biosearch

TABLE 2

| | Blue Region | Green Region | Red Region |
|---|---|---|---|
| Name | [nm] | [nm] | [nm] |
| Hematoporphyrin Derivative (HPD) | 380-420 (405) | 490-520 | 600-670 |
| | | (502) | (630) |
| Photofrin II (PII) | 380-420 (405) | 490-520 | 600-670 |
| | | (502) | (630) |
| Tetra (m-hydroxyphenyl) | 400-450 | 500-560 | 600-680 |
| chlorin (mTHPC) | (420) | (520) | (652) |
| Benzoporphyrin Derivative Mono Acid | 400-460 (430) | | 600-670 |
| Ring (BPD-MA) | | | (630) |
| | | | 670-720 |
| | | | (690) |
| Zinc-Phthalocyanin | 320-400 (343) | | 580-630 |
| (ZnPC) | | | (607) |
| | | | 650-700 |
| | | | (671) |
| Protoporphyrin IX | 380-440 | | 600-680 |
| | (405) | | (635) |
| Chlorin e6 | 380-440 | | 600-690 |
| | (410) | | (662) |
| AlS4Pc | 320-400 (343) | | 580-630 |
| | | | (607) |
| | | | 650-700 |
| | | | (671) |
| Texaphyrins | 400-500 | | 690-780 |
| | 450 | | (732) |
| Hypericin | 400-500 | 520-600 | 570-650 |
| | (475) | (550) | (592) |
| Pheophorbide a | 350-450 | | 600-720 |
| | (400) | | (670) |

The wavelengths given in brackets are the maxima (±5 nm) of the particular absorption band.
Technologies, USA). The amine group was protected with a trifluoroacetyl (TFA) protecting group. A TFA protected Aminohexyl Amidite (5′TFA-Amino C6 Modifier, Biosearch Technologies, USA) was used to produce a functional amine group on the 5′ end of the oligonucleotide. The CPG support was treated with 28% ammonium hydroxide at 55° C. for 8 hours following this the ammonium hydroxide solution was evaporated in vacuo.

The N-hydroxy succinimide (NHS) esters of both photosensitive moieties (pheophorbide a and chlorin e6 (Porphyrin Products, Logan, Utah) were prepared by reacting 1.5 equivalents of dicyclohexylcarbodiimide and 1.5 eqivalents of NHS with 1 equivalent of chlorin e6 and pheophrbide a in dry dimethylsulfoxide in the dark for 24 hours at room temperature. The dry, crude oligonucleotide (20-250 nmols) was dissolved in sodium bicarbonate buffer (0.1 M, pH 9.3, 500 μl) and 20 equivalents of the corresponding photosensitive moiety NHS ester (dissolved in 100 μl DMSO) were added in 10 μl aliquots at 20 minute intervals to the mixture. After continuous stirring for 24 hours at room temperature, particulate matter was removed by spinning the mixture in a microcentrifuge for one minute at 10000 rpm. In order to remove unreacted photosensitive moiety, the supernatant was passed through a gel-exclusion column, equilibrated with 0.1 M triethylammonium acetate at pH 6.5. The resulting Ω-sensitizers were purified using a C-18 reverse phase column (Waters) HPLC utilizing a linear elution gradient of 20 to 70% of 0.1 M triethylammonium acetate in 75% acetonitrile in 0.1 M triethylammonium acetate. HPLC was monitored at 280 nm and 665 nm. Fractions having peaks that at both wavelengths were collected, precipitated with ethanol and salt. After drying the Ω-sensitizers, 5′-(pheo a)-GCG ATG CTA GGT TTC CTC CCT TTG TCG C-(pheo a)-3′ and 5′-(chlorin)-GCG ATG CTA GGT TTC CTC CCT TTG TCG C-(chlorin)-3′, respectively, were ready to use.

In a similar manner as described above control random Ω-sensitizers with the scrambled sequence 5′-GCG ACT GAC TGC CAA CTA TGA ACA TCG C-3′ conjugated to pheophorbide a and chlorin e 6 were synthesized. The nucleic acid sequence 5′-CTG ACT GCC AAC TAT GAA CA-3′ has been chosen because it has been shown to be GenBank negative for any known RNA match.

### EXAMPLE 2

The selective photosensitizing action of the different Ω-sensitizers described in Example 1 was tested in vitro against sense oligonucleotides, and sense oligonucleotides having one and two base mismatches, respectively. For this purpose oligonucleotides with sequences 5′-CAA AGG GAG GAA ACC TAG CA-3′ (sense), 5′-CAA AGG GAG GTA ACC TAG CA-3′ (sense 1 bp mismatch), and 5′-CAA AGG GAA GTA ACC TAG CA-3′ (sense 2 bp mismatch) were synthesized using standard automated phosphoramidite chemistry. Oligonucleotides were dissolved in 2 ml of a solution containing 10 mM Tris-HCl and 2 mM MgCl₂ to give a final concentration of 0.8 μM in a quartz cuvette. To this solution 1 ml Dihydrorhodamine 123 (DHR123) (Molecular Probes, Eugene, USA) (40 μM) dissolved in 10 mM Tris-HCl was added. DHR123 is a non-fluorescent molecule that in the presence of oxygenating species undergoes oxygenation to give the fluorescent dye rhodamine 123 having an absorption maximum at 507 nm and a fluorescence maximum at 529 nm. 1 ml of Ω-sensitizer (800 nM) containing solution was added and the final solution was stored in the dark at 37° C. for 20 minutes. Then the solution was irradiated at 405±5 nm with electromagnetic radiation from a filtered Hg-Arc lamp. The RH123 fluorescence intensity at 529 nm was measured every 60 seconds using a Perkin Elmer spectrofluorometer (Model LS50B, Perkin Elmer, Rotkreuz, CH)

FIG. 7 shows the RH123 fluorescence intensity-electromagnetic radiation dose profiles for pheophorbide a (FIG. 7A) and chlorin e6 (FIG. 7B) labeled Ω-sensitizers. In both cases the most efficient photochemically induced oxidation of DHR123 was observed in the presence of the sense oligonucleotide (squares). One base pair mismatches (dots) and two base pair mismatches (up-triangles) resulted in about 5-fold and 20-fold reduction of oxidation capacity, respectively. The control Ω-sensitizers exhibited only a very reduced photo-oxidation (down triangles) in the presence of the sense oligonucleotide. Since pheophorbide a is a slightly more potent photosensitive moiety than chlorin e6, and its photobleaching rate is slightly lower than chlorin e6, higher fluorescence intensities were achieved with the pheophorbide a labeled Ω-sensitizer.

Furthermore, the intrinsic fluorescence of the pheophorbide a labeled Ω-sensitizer was used to further underline the selectivity of these new photochemotherapeutic agents. FIG. 8 shows the fluorescence emission spectrum after excitation of the Ω-sensitizer at 405 nm in the presence of the sense oligonucleotide (straight line), the one base pair mismatch sense oligonucleotide (dashed curved), and the two base pair sense mismatch oligonucleotide (dotted line). From FIG. 8 it can be seen that oligonucleotides having two base pair mismatches resulted in a significant reduction of the fluorescence of the photosensitiser pheophorbide a.

### EXAMPLE 3

In order to test the possibility of using nano particles as the quenching moiety, oligonucleotides labeled with a photosensitive moiety at the 3′ terminus, where labeled at the 5′ terminus with amino modified gold nanoparticles. The mam gene sequence S′-CGG ATG AAA CTC TGA GCA ATG TCT GCA GTT CTG TGA GCC AAA G-3′ (GeneBank accession No. AF015224) was coupled to the complementary stem sequences CCA AGC and GCT TGG at its 5′ and 3′ termini, respectively. The sequence was constructed using fully automated DNA synthesis as described in Example 1. The 3′ end contained an amino group, while the 5′ end was equipped with a six carbon spacer thiol group protected by a trityl moiety (Glen Research, Sterling, Va.) using standard methods. Following cleavage with 28% ammonium hydroxide, oligonucleotides were washed and purified as described above. The 3′ end photosensitive moiety labeled oligonucleotide was prepared and purified using the NHS ester of pheophorbide a as described in Example 1. Then the trityl protection group was removed by adding 0.15M silver nitrate solution to the labeled oligonucleotide for 1 hour. To this mixture 0.15M dithiothreitol (DTT) was added for 10 minutes. After spinning at 10000 rpm the supernatant was transferred to another tube. 400 pmol of this oligonucleotide were then suspended in 180 μl of water and immediately reacted with 6 nmol of monomaleimido gold particles (Ø=1.4 nm) (Nanoprobes) in aqueous 20 mM NaH₂PO₄, 150 mM NaCl, 1 mM ethylenediamine tetraethyl acetate (EDTA) Buffer, pH 6.4, containing 10% isopropanol at 4° C. for 24 hours.

To the solution containing the Ω-sensitizer, 5′ (gold particle)-S—(CH₂)₆— CCA AGC CGG ATG AAA CTC TGA GCA ATG TCT GCA GTT CTG TGA GCC AAA GCT TGG-(pheophorbide a)-3′, target and one base pair mismatch oligonucleotides were added at different concentrations. After adding appropriate quantities of DHR123 (see Example 1), the solutions were irradiated at 405 nm with electromagnetic radiation doses of 10 J/cm² and the fluorescence of rhodamine 123 was measured as described above as a function of the target concentration. FIG. 9 shows the concentration dependence of Ω-sensitizer-induced photo-oxidation of DHR123 as measured by means of RH123 fluorescence at 529 nm in the presence of a target sequence (squares) and in the presence of a one base pair mismatched oligonucleotide (circles). The fluorescence intensity in the presence of the target sequence reaches its maximum at a concentration of about 0.5 μM of the target sequence, while no fluorescence is detectable at this concentration when the oligonucleotide is not complementary to the TCS. At only very high concentrations of the one base pair mismatched oligonucleotide can photodynamic action be observed.

### EXAMPLE 4

In the case of breast cancer cell lines there is considerable evidence of altered integrin level expression in the tumorigenic situation in comparison with the native, parental background. AlphaV integrins are highly expressed and could have a prominent role in breast cancer metastasis to bone. Recently, cDNA sequences encoding the integrin alphaV from a variety of species were assessed using DNA Star and GCG molecular biology packages. From a number of sequences one 18 bp target region spanning nucleotide positions 40-57 in the human DNA was chosen. A modified Ω-sensitizer containing a quencher moiety on the 5′ terminus and a photosensitizing moiety on the 3′ terminus was produced. The corresponding alphaV 5543-ODN TCS sequence 5′-GC*G AG*C* GGC*GGA AAA GC*C A*T*C *GTC*G C-3′ and the mismatch control sequence 5′-GC*G AG*C* AAC*GAG AGA GC*C G*T*C* GTC*G C-3′ were synthesised as described previously (*represents phosphorthioate linkages). The 3′ terminus was additionally modified using a C6 thioether modifier as described above. All oligonucleotides were routinely synthesized as partial phosphorothioates under standard conditions. Oligonucleotides were synthesized using an Applied Biosystem 394 DNA synthesizer (Perkin Elmer Applied Biosystems, Foster City, Calif.) and standard phosphoramitide chemistry. After coupling, phosphorthioate linkages were introduced by sulphorization using Beaucage reagent, followed by capping with acetic anhydride and N-methylimidazole. After cleavage from the solid support and final deprotection by treatment with concentrated ammonia, oligonucleotides were purified by polyacrylamide gel electrophoresis. Following purification chlorin e6 NHS ester was introduced as described in Example 1 at the 3′ end. Then the trityl protection group was removed by adding 0.15M silver nitrate solution to the labeled oligonucleotide for 1 hour. To this mixture 0.15 M dithiothreitol (DTT) was added for 10 minutes. After spinning at 10000 rpm the supernatant was transferred to another tube. 400 pmol of this oligonucleotide were then suspended in 180 μl of water and immediately reacted with QSY-7 maleimide (3.6 mg) (Molecular Probes, Eugene, USA) and subsequently dissolved in DMF for 10 minutes at 65° C. Unreacted QSY-7 was removed with spin column chromatographic separation using a Sephadex G-25 spin column. After purification, Ω-sensitizers were incubated with target oligonucleotide (sequence 5′-CGA TGG CTT TTC CGC CGC-3′) in a quartz cuvette and the intrinsic fluorescence of the photosensitive moiety moiety was followed at 664 nm for 20 minutes. FIG. 10 shows the fluorescence time profiles of the hybridization of the antisense and control Ω-sensitizer in the presence of the alphaV target sequence in the solution. Only slightly increasing fluorescence was observed with the random control Ω-sensitizer, while the antisense Ω-sensitizer developed within 25 minutes maximal fluorescence intensities due to hybridization to the target sequence. This indicates that treatment of cells expressing the alphaV integrin target cDNA will have maximal efficacy after about 30 minutes following incubation.

### EXAMPLE 5

In order to test for the phototoxic potential of the Ω-sensitizer of Example 4, cells encoding the target were treated with the Ω-sensitizer, with the Ω-sensitizer and electromagnetic radiation, and with the TCS only. The therapeutic outcome was assessed by a cell adhesion assay.

Cells

The MDA-MB231 human breast carcinoma cell line was purchased from the European Tissue Culture Collection (ETCC). Cells were cultured in Dulbecco's Modified Minimum Essential Medium (DMEM) with Earle's salts containing 10% fetal calf serum (FCS), 100 U/ml penicillin 0.2 mg/ml streptomycin, 0.2% glycine at 37° C. in 5% CO₂, 95% air in a humidified atmosphere.

Treatment

Cells were plated in 24-well multiwell dishes and incubated with the oligonucleotides at different concentrations. Incubation was performed in the presence of the uptake enhancer lipofectamine (Gibco, Life Technologies, Paisley, UK) (5 μg/ml). 60 minutes after incubation the cells were irradiated at 410 nm with electromagnetic radiation doses ranging between 0 and 6 J/cm². Then, twenty-four hours following treatment, wells were rinsed three times with PBS to remove non-adhering cells. Each well was then treated with 20% methanol (10 min), and the cells were stained with 0.5% crystal violet in 20% methanol before rinsing with distilled water and subsequently air-dried for 15 minutes. The crystal violet was then solubilized with 100 μl of 0.1N sodium citrate in 50% ethanol and transferred to 96-well mictrotitre plates, and absorbance, which was linearly proportional to the number of attached cells, read spectrophotometrically at 540 nm.

Results

FIG. 11 shows the percentage of attached cells (versus non-treated cells) under different treatment conditions, 24 hours after treatment with an antisense oligonucleotide (squares), the targeting Ω-sensitizer without irradiation (dots), a control Ω-sensitizer without irradiation (up triangles), the control Ω-sensitizer after irradiation with a electromagnetic radiation dose of 2 J/cm² (diamonds) and the targeting Ω-sensitizer after irradiation with a electromagnetic radiation dose of 2 J/cm² (down triangles). It can be seen from FIG. 11 that the treatment with both the antisense and the targeting Ω-sensitizer resulted in a considerable reduction of cell adherence during the 24 hour incubation. While the control Ω-sensitizer resulted in no significant modulation of cell adherence under experimental conditions, the cells treated with 0.01 μM of the Ω-sensitizer followed by irradiation with electromagnetic radiation at 410 nm at a electromagnetic radiation dose of 2 J/cm² reduced the number of adherent cells by about 60%. Higher drug doses nearly totally suppressed the adherence of the breast cancer cells. Furthermore, the loss of adherence was dependent on the electromagnetic radiation dose given to the cells in the presence of 0.1 μM of the targeting Ω-sensitizer (FIG. 12, squares), which was not observed when the control Ω-sensitizer was used (FIG. 12, dots)

### EXAMPLE 6

To test the possibility of inhibiting cell growth by targeting the EGFR mRNA with Ω-sensitizers, mixed backbone oligonucleotides similar to those described in Example 1 were prepared, using methods described in Example 5. The sequences tested were:

| | |
|---|---|
| a) | 5′ (chlorin e6) -GC*G A*T*G CTA GG*T* TTC CTC |
| | CCT T*T*G* T*C*G C- (chlorin e6) 3′ |
| b) | 5′ (chlorin e6) -GC*G A*CT GA*C TGC CA*A C*TA |
| | TG*A* ACA T*C*G C-(chlorin e6)3′ |
| c) | 5′ -*T*G CTA GG*T* TTC CTC CCT T*T*G*-3′ |

(as previously described * represents phosphorthioate linkages)

Cells

GEO human colon cancer cells known to express approximately about 40000 functional EGFRs were purchased from ATCC (Rockville, Md., USA) and grown as described. Culture was performed in the presence of 10% fetal calf serum (FCS) and penicillin/streptomycin at 37° C. and 6% CO₂ in a humid environment. For measurement purposes, the cells were subcultured in 48-well dishes (Costar 3548, Integra Biosiences, Cambridge, Mass., USA) to give 10⁵ cells/well 72 hours prior to incubation with the oligonucleotides. Cells were incubated with the oligonucleotides for 60 minutes and irradiated at 405±5 nm with the electromagnetic radiation of a filtered Hg-Arc lamp with a electromagnetic radiation dose of 5 J/cm².

Determination of Cell Viability

The cell viability was tested by means of an MTT assay. This technique allows quantification of cell survival after cytotoxic insult by testing the enzymatic acitivity of the mitochondria. It is based on the reduction of the water-soluble tetrazolium salt to a purple, insoluble formazan derivative by the mitochondrial enzyme dehydrogenase. This enzymatic function is only present in living, metabolically active cells. The optical density of the product was quantified by its absorption at 540 nm using a 96-well ELISA plate reader (iEMS Reader MF, Labsystems, USA). MTT, 0.5 mg/ml, was added to each well 24 hours after irradiation and incubated for 2 hours at 37° C. The medium was then removed and the cells were washed with PBS. Cell lysis and dissolution of the formazan crystals formed was achieved by the addition of 2501 of isopropanol containing 1% 4N HCl. The absorption of each residue was determined at 540 nm using the plate reader. The absorbance readings from treated cells was divided by the absorbance readings from the control cells in order to calculate the fraction of surviving cells.

Results

FIG. 13a shows the inhibition of the mitochondrial activity of the cells one day after incubation with the oligonucleotides in the dark. Both oligonucleotides complementary to the EGFR mRNA were able to significantly reduce cell viability (circles, squares), while cell viability was only slightly reduced using the control sequence (up triangles). However, irradiation with electromagnetic radiation at an electromagnetic radiation dose of 5 J/cm² significantly reduced cell viability only when using the targeting Ω-sensitizer (circles) at drug doses that are in the order of two orders of magnitude lower than without irradiation (FIG. 13B).

1. A compound having a structure selected from the group consisting of X—Rₙ-A-Qₘ-Y, Rₙ—X-A-Y-Qₘ, Rₙ—X-A-Qₘ-Y and X—Rₙ-A-Y-Qₘ wherein,
A is a single-stranded nucleic acid sequence, said single-stranded nucleic acid sequence being complementary to a pre-selected target sequence;
R is a photosensitive moiety such that upon irradiation with electromagnetic radiation having a wavelength corresponding to at least one absorption wavelength of R, R interacts through energy transfer with a molecule capable of producing free radicals, to produce free radicals;
Q is a moiety that quenches excited energy states of R;
X and Y are an affinity pair that interact to bring R and Q into close proximity in the absence of said target sequence thus enabling energy transfer between R and Q;
n and m are, independently, integers in the range 1-5; and
said compound optionally contains a linker moiety.

2. The compound according to claim 1, wherein R has an absorption wavelength of between 300 and 800 nm.

3. The compound according to claim 1, wherein R is selected from the group consisting of chlorines, chorophylls, coumarines, cyanines, fullerenes, metallophthalocyanines, metalloporphyrins, methylenporphyrins, naphthalimides, naphthalocyanines, nile blue, perylenequinones, phenols, pheophoribes, pheophyrins, phthalocyanines, porphycenes, porphyrins, psoralens, purpurins, quinines, retinols, rhodamines, thiophenes, verdins, xanthenes and dimers, oligomers and derivatives thereof.

4. The compound according to claim 1, wherein Q is selected from the group consisting of a non-fluorescing dye, a fluorophore, a second photosensitizing moiety, a nano-scaled semiconducter or conductor and gold.

5. The compound according to claim 4, wherein the second photosensitizing moiety is different than R.

6. The compound according to claim 1, wherein X and Y are selected from the group consisting of complementary nucleic acid sequences, protein-ligand, antibody-antigen and protein-nucleic acid.

7. The compound according to claim 1, wherein the linker moiety is selected from the group consisting of linear substituted alkyl, linear unsubstituted alkyl, branched substituted or alkyl) branched unsubstituted alkyl, linear substituted heteroalkyl, linear unsubstituted heteroalkyl, branched substituted heteroalkyl, and branched substituted heteroalkyl groups.

8. The compound according to claim 1, wherein said molecule capable of producing free radicals is molecular oxygen.

9. The compound according to claim 8, wherein said free radicals are selected from the group consisting of singlet oxygen and reactive oxygen species.

10. The compound according to claim 1, wherein the compound is unimolecular.

11. The compound according to claim 1, wherein the compound is bimolecular.

12. A complex comprising a compound according to claim 1 which is bound to a carrier which increases the internalization of said compound.

13. The complex according to claim 12, wherein said compound is bound to said carrier by electrostatic interaction or covalent interaction.

14. The complex according to claim 13, wherein the carrier is a polycation.

15. The complex according to claim 14, wherein the polycation is a histone or polylysine.

16. The complex according to claim 12, wherein said compound is bound to said carrier by covalent interaction.

17. The complex according to claim 13, wherein the carrier is a protein or peptide.

18. The complex according to claim 17, wherein the protein is an antibody, an antibody fragment, or a cholesterin.

19. The complex according to claim 12 wherein the carrier targets a specific cell surface protein.

20. The complex according to claim 19, wherein the cell surface protein is selected from the group consisting of a low-density lipoprotein receptor, an endothelial growth factor receptor, a fibroblast growth factor receptor, an integrin, an insulin receptor, an epidermal growth factor receptor and a transferrin receptor.

21. The complex according to claim 12, wherein the complex is encapsulated in a lipid mixture, said lipid mixture comprising at least two members independently selected from the group consisting of phospholipids, sterols and cationic lipids.

22. The complex according to claim 21, wherein the lipid mixture comprise liposomes.

23. The complex according to claim 22, wherein the liposomes are from about 50 to 150 nm in diameter.

24. A pharmaceutical composition comprising (a) a compound according to claim 1, wherein said compound is optionally bound to a carrier which increases the internalization of said compound and (b) at least one pharmaceutically acceptable carrier or excipient.

25-29. (canceled)

30. A method of killing cells by photochemotherapy comprising the steps:
(i) incubating target cells with an effective amount of a compound according to claim 1, optionally bound to a carrier which increases the internalization of said compound;
(ii) allowing sufficient time for the compound to hybridise to a target nucleic acid sequence within the cells; and
(iii) irradiating the target cells with electromagnetic radiation of a wavelength that corresponds to at least one absorption wavelength of the photosensitive moiety R such that R interacts through energy transfer with a molecule capable of producing free radicals, to produce free radicals which kill said cells.

31. The method according to claim 30, wherein said molecule capable of producing free radicals is molecular oxygen.

32. The method according to claim 31, wherein said free radicals are selected from the group consisting of singlet oxygen and reactive oxygen species.

33. The method according to claim 30, wherein irradiation with electromagnetic radiation is performed within between 1 minute and 168 hours after incubation with the compound.

34. The method according to claim 30, wherein the total fluence of electromagnetic radiation used for irradiation is between 2 J/cm² and 500 J/cm².

35. A kit for preparing a compound according to claim 1 comprising:
(a) one or more affinity pairs;
(b) one or more photosensitizing and quenching moieties; and
(c) one or more target complement sequences.

36. (canceled)

37. The method according to claim 30 which is useful for treating a patient suffering form a condition selected from the group consisting of neovascularization, age related macular degeneration, diabetic retinopathy, arthritis, and cancer.
